(19) United States
(12) Patent Application Publication
(10) Pub. No.: US 2026/0256440 A1
(43) Pub. Date: Sep. 3, 2026

(54) METHOD AND APPARATUS FOR PROCESSING IMAGE DATA IN AN X-RAY IMAGING SYSTEM

(71) Applicant: CANON KABUSHIKI KAISHA, Tokyo (JP)

(72) Inventors: Joseph F. WHITEHEAD, Vernon Hills, IL (US); Joseph J. MANAK, Vernon Hills, IL (US); Yi HU, Vernon Hills, IL (US)

(73) Assignee: CANON KABUSHIKI KAISHA, Tokyo (JP)

(21) Appl. No.: 19/067,561

(22) Filed: Feb. 28, 2025

(51) Int. Cl. A61B 6/00; A61B 6/06; G06T 7/00; G06T 11/00

(52) U.S. Cl. CPC A61B 6/5205; A61B 6/06; G06T 7/0012; G06T 12/10; G06T 12/20; G06T 2207/10081; G06T 2207/10116; G06T 2207/20081; G06T 2207/20084; G06T 2207/30004; G06T 2210/41

(57) ABSTRACT

A method for performing image processing in an X-ray imaging system having a collimator. The method includes obtaining a 2D projection image acquired by the X-ray imaging system, and inputting the obtained image into a pretrained machine-learning model to infer an image. The inferred image has an image quality better than that of the obtained image. The pretrained machine-learning model is trained on a plurality of training images. Each training image is obtained by receiving a first 2D projection image acquired without a collimator, determining a collimator simulation parameter, estimating a scatter field for the first 2D projection image, generating a noise-simulating image based on the estimated scatter field, and generating a penumbra-simulating image to simulate a penumbra caused by a collimator, based on the determined collimator simulation parameter, and generating a second 2D projection image by combining the noise-simulating image, the penumbra-simulating image, and the first 2D projection image.

### BACKGROUND

### Field

This disclosure relates to medical imaging techniques, including, but not limited to, 2D projection X-ray imaging, Computed Tomography (CT) imaging, C-arm interventional imaging, etc.

### Description of the Related Art

Deep learning-based image processing methods have demonstrated superior performance over classical techniques across various medical imaging modalities. For instance, artificial intelligence (AI) algorithms can be trained to perform a variety of corrections for X-ray images acquired from C-arm interventional systems, such as 2D and 3D denoising, truncation correction, saturation correction, cone-beam artifact correction, etc.

Image data for training such AI algorithms can be obtained from clinical C-arm systems, for example. However, the input training data is often acquired without collimated field-of-views (FOVs). As a result, the algorithm is trained on data that does not have collimators in the FOVs. Consequently, when applied to collimated image data, the algorithm may generate artifacts.

Therefore, there is a need for an improved approach for training neural networks in medical imaging systems to enhance the image quality and ensure robust performance across varying imaging conditions.

### SUMMARY

The present disclosure relates to a method for performing image processing in an X-ray imaging system. The X-ray imaging system has a collimator for collimating an incident direction of X-rays with respect to an X-ray detector. The method includes obtaining a two-dimensional (2D) projection image acquired by the X-ray imaging system, and inputting the obtained 2D projection image into a pretrained machine-learning model, to infer a 2D projection image from an output of the pretrained machine-learning model. The inferred 2D projection image has an image quality better than an image quality of the obtained 2D projection image. The pretrained machine-learning model is trained on a plurality of training images. Each training image of the plurality of training images is obtained by receiving a first 2D projection image acquired by an X-ray imaging system under a condition that no collimator is applied for X-ray collimation, determining a collimator simulation parameter, estimating a scatter field for the first 2D projection image, generating a noise-simulating image based on the estimated scatter field, and generating a penumbra-simulating image to simulate a penumbra caused by a collimator, based on the determined collimator simulation parameter, and generating a second 2D projection image, as the training image, by combining the noise-simulating image, the penumbra-simulating image, and the first 2D projection image.

The disclosure additionally relates to an apparatus for performing image data processing in an X-ray imaging system. The X-ray imaging system has a collimator for collimating an incident direction of X-rays with respect to an X-ray detector. The apparatus includes processing circuitry configured to obtain a 2D projection image acquired by the X-ray imaging system, and input the obtained 2D projection image into a pretrained machine-learning model, to infer a 2D projection image from an output of the pretrained machine-learning model. The inferred 2D projection image has an image quality better than an image quality of the obtained 2D projection image. The pretrained machine-learning model is trained on a plurality of training images. Each training image of the plurality of training images is obtained by receiving a first 2D projection image acquired by an X-ray imaging system under a condition that no collimator is applied for X-ray collimation, determining a collimator simulation parameter, estimating a scatter field for the first 2D projection image, generating a noise-simulating image based on the estimated scatter field, and generating a penumbra-simulating image to simulate a penumbra caused by a collimator, based on the determined collimator simulation parameter, and generating a second 2D projection image, as the training image, by combining the noise-simulating image, the penumbra-simulating image, and the first 2D projection image.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIGS. 1A and 1B show images processed with an artificial intelligence (AI) denoising algorithm at different window levels, where the denoising algorithm is trained on images acquired without collimators in the field-of-views (FOVs);

FIG. 2 shows a block diagram of an exemplary image data processing apparatus 200 in accordance with embodiments of the disclosure;

FIG. 3 shows a flow chart of an exemplary procedure 300 for performing image data processing in accordance with embodiments of the disclosure;

FIG. 4 shows a block diagram of exemplary training dataset generating circuitry 210 in accordance with embodiments of the disclosure;

FIGS. 5A, 5B, and 5C show an original 2D projection image without collimator simulation and images with collimator simulation at different window levels;

FIGS. 6A and 6B show images processed with an AI denoising algorithm at different window levels, where the denoising algorithm is trained on images generated with collimator simulation in accordance with embodiments of the disclosure;

FIG. 7 shows a flow chart of an exemplary procedure 600 for generating a training dataset in accordance with embodiments of the disclosure;

FIG. 8 shows a schematic block diagram of an exemplary X-ray diagnostic system that can incorporate the techniques disclosed herein;

FIG. 9 is a schematic of an implementation of the X-ray diagnostic system illustrated in FIG. 8; and

FIG. 10 is a block diagram illustrating an exemplary computer system for implementing the embodiments described in this disclosure.

### DETAILED DESCRIPTION

The following disclosure provides embodiments or examples for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,” unless stated otherwise.

Artificial intelligence (AI) algorithms have been used across various X-ray imaging modalities to enhance the image quality. However, as discussed previously, when a neural network is trained on image data that is acquired without collimators in the field-of-views (FOVs), and then applied to collimated image data, the differences in the imaging conditions may lead to artifacts.

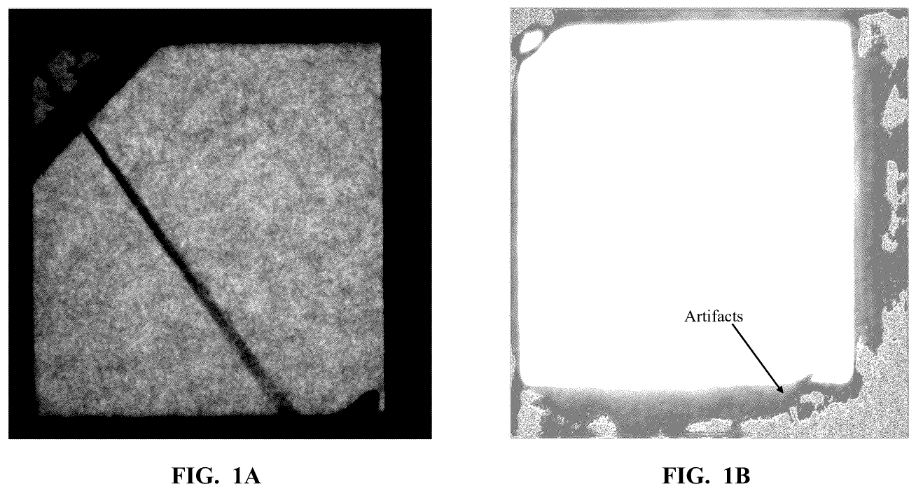

FIGS. 1A and 1B illustrate, at different window levels, images inferred by a denoising network. As the denoising network is trained on images acquired without collimators in the FOVs and later applied to images acquired with a collimator, visible artifacts are introduced due to the inconsistencies in imaging conditions, as seen more apparently in FIG. 1B. Note that the images of FIGS. 1A and 1B are obtained prior to dynamic range compression in the imaging chain. After the dynamic range compression, the artifacts will become even more pronounced.

The present disclosure provides a collimator simulation approach to enhance the robustness of AI image processing algorithms. By simulating the effects of collimators on 2D projection images, the approach provides a form of data augmentation for the training of 2D image processing neural networks. By generating sufficiently realistic collimator simulations that simulate collimator effects controlling the FOVs, the performance of AI algorithms on real collimated image data can be improved.

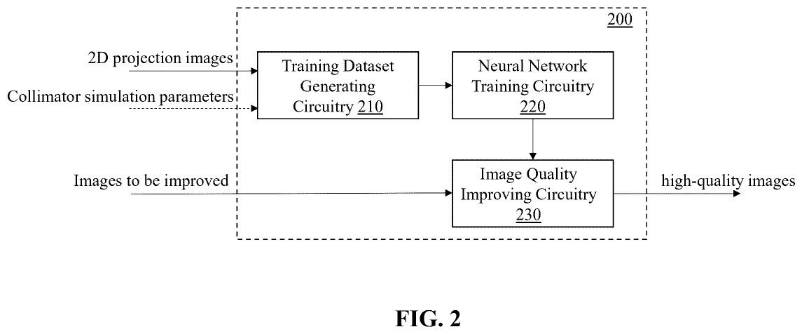

FIG. 2 shows a block diagram of an exemplary image data processing apparatus 200 in accordance with embodiments of the disclosure. The processing apparatus 200 includes training dataset generating circuitry 210, neural network training circuitry 220, and image quality improving circuitry 230.

The training dataset generating circuitry 210 receives 2D projection images that are acquired without collimators in the FOVs. The 2D projection images can be collected from various sources, such as research experiments on phantoms and volunteers, clinical procedures performed on patients, etc.

Additionally, the training data set generating circuitry 210 receive collimator simulation parameters that define one or more characteristics of the collimators to be simulated. For example, these parameters can be specified by an operator user of the image data processing apparatus 200.

While FIG. 2 shows that the training dataset generating circuitry 210 receives the collimator simulation parameters, some or all of the collimator simulation parameters can be randomly selected by the training data set generating circuitry 210 from predetermined value. This approach can further enhance the generalizability of the neural network to be trained.

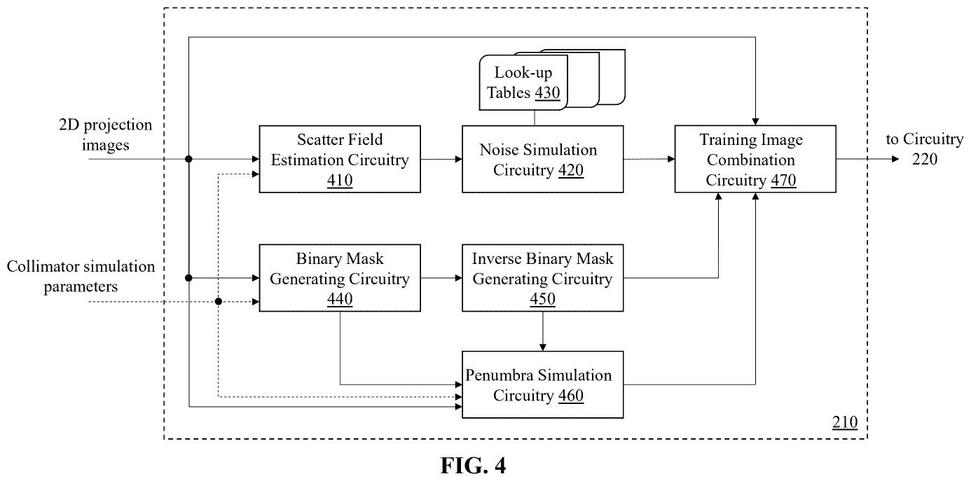

Based on the 2D projection images and the collimator simulation parameters, the training dataset generating circuitry 210 generates a training dataset and sends it to the neural network training circuitry 220. The structure and functionality of the training dataset generating circuitry 210 will be described below with reference to FIGS. 4-6.

The neural network training circuitry 220 uses the training dataset generated by the training dataset generating circuitry 210 to train a neural network. The neural network can be a denoising network, a deblurring network, or a network trained to remove artifacts in images, for example.

Once the network parameters are determined through training, the neural network can function as the image quality improving circuitry 230. It receives, as an input, 2D projection images acquired by a medical imaging system having a collimator. The input images typically exhibit a lower image quality, and the neural network can be applied to process them to generate output images with an improve image quality.

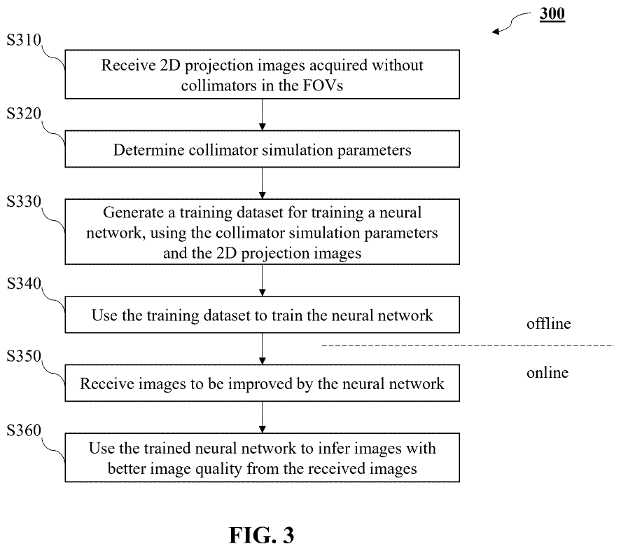

FIG. 3 shows a flow chart of an exemplary procedure 300 for performing image data processing in accordance with embodiments of the disclosure. The procedure 300 includes an offline portion (steps S310-S340) and an online portion (steps S350-S360).

In step S310, 2D projection images acquired without collimators in the FOVs are received. In step S320, a set of collimator simulation parameters are determined. In step S330, a training dataset with collimator simulation is generated for training a neural network to be used in image quality enhancement. In step S340, the neural network is trained using the generated training dataset. In step S350, lower-quality images acquired by a medical imaging system with a collimator are received. In step S360, the trained neural network infers higher-quality images from the received lower-quality images.

In X-ray imaging, both primary (unscattered) photons and scattered photons can reach the detector. Particularly, when the imaging object is thick or wide, the number of scattered photons increases due to more frequent photon-material interactions. The scattered photons introduce noise into the 2D projection images. Even in the regions shielded by the collimator, the detected signal is not uniformly zero because of the scattered photons. When simulating the collimator effects in a 2D projection image, it is essential to account for the scatter and the resulting noise.

Furthermore, images acquired with a collimator often exhibit penumbra effects at the edges of the collimator. The penumbra can result from increased signals at the collimator edges caused by scattered photons or the infinite size of the focal spot. For a more realistic simulation of collimator effects, it is necessary to incorporate the penumbra in the images.

FIG. 4 shows a block diagram of exemplary training dataset generating circuitry 210 in accordance with embodiments of the disclosure. The training dataset generating circuitry 210 includes scatter field estimation circuitry 410, noise simulation circuitry 420, a look-up table storage 430, binary mask generating circuitry 440, inverse binary mask generating circuitry 450, penumbra simulation circuitry 460, and training image combination circuitry 470.

The scatter field estimation circuitry 410 receives a 2D projection image acquired without a collimator, and estimate a scatter field. Various methods can be used for scatter estimation. For example, an analytical partial differentiation equation, e.g., a radiative transfer equation, can be solved to determine the scatter distribution. Alternatively, a Monte Carlo approach can be used to estimate the scatter signal by simulating individual photon interactions as they pass through the imaging object. Since no real 3D object data is available at this stage, a 3D phantom can be used instead, such as computational human phantom (XCAT), a water cylinder, or another anthropomorphic phantom.

As another example, a neural network pretrained on known scatter patterns can predict scatter for the 2D projection image. The neural network can be trained on a dataset that includes diverse scatter scenarios to ensure accuracy of the scatter estimation. Compared with the Monte Carlo method, the neural network method can provide significantly faster scatter estimation.

To simplify the scatter estimation process, a constant scatter signal can be assumed. As shown in FIG. 4, based on a collimation simulation parameter representing a scaling factor, the scatter field estimation circuitry 410 can estimate the scatter signal as follows:

$${I}_{s} = mean ( I ) \times α \qquad ( 1 )$$

wherein I denotes the input 2D projection image, Iₛ denotes the estimated scatter signal, mean (I) represents the mean intensity of the input image. The scaling factor a can be specified by the user or randomly selected by the scatter field estimation circuitry 410 from a group of predetermined values.

The predetermined values of a can be derived using various methods. For example, a values can be calculated by measuring scatter signals in collimated images across different image intensities and object thicknesses/widths. Additionally, Monte Carlo simulations can be performed to simulate the scatter and determine the a values.

Once the scatter signal Iₛ is estimated, the noise simulation circuitry 420 can generate a corresponding noise image I_{s, noise}, which simulates the noise caused by the scatter in the image. In the example shown in FIG. 4, the relationship between scatter and noise can be measured on the imaging system used to acquire the 2D projection image and stored as look-up tables. These look-up tables provide a model of how scatter contributes to noise.

By referencing the look-up tables stored in the look-up table storage 430, the noise simulation circuitry 420 generates the noise image I_{s, noise}. The noise image I_{s, noise} has the same size as the input 2D projection image, and contains noise simulated based on the scatter signal Iₛ.

Although the example shown in FIG. 4 focuses on simulating noise caused by scatter, other types of noise can also be simulated using the look-up table method or through a Monte Carlo-based approach. These types of noise can include electronic noise, noise from a polychromatic beam and detector gain, for example.

Based on a collimator simulation parameter that defines the percentage of the image area to be collimated, the binary mask generating circuitry 440 generates a binary mask image I_{B}. The binary mask image I_{B} has the same size as the input 2D projection image, with zeros at positions not covered by the collimator and ones at positions where the collimator is applied. The collimator simulation parameter defining the percentage of collimation can be specified by the user, or randomly selected by the binary mask generating circuitry 440 from a set of predetermined values.

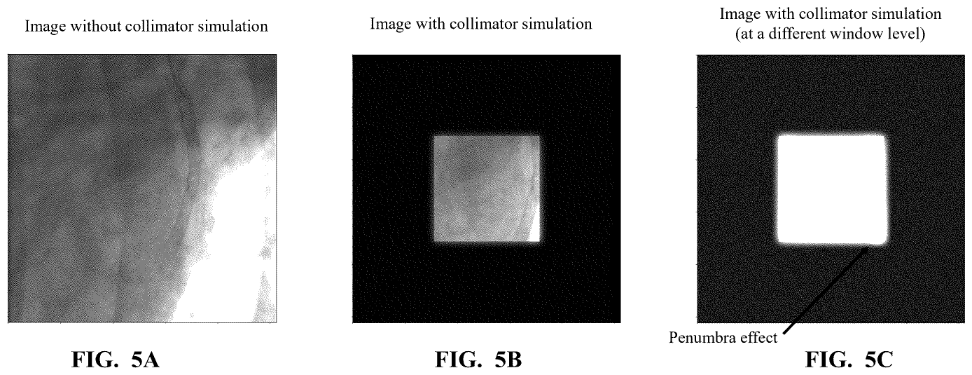

FIGS. 5A, 5B, and 5C show an original 2D projection image without collimator simulation (FIG. 5A) and images with collimator simulation (FIGS. 5B and 5C, at different window levels). The images in FIGS. 5B and 5C simulate the effects of a square collimator; however, a rectangular collimator also can be simulated. For example, an additional collimator simulation parameter can define the aspect ratio between the row direction and column direction of the detector. This parameter can be specified by the user, or randomly selected by the binary mask generating circuitry 440 from a set of predetermined values, allowing for asymmetric collimator simulation along the row and column directions.

After generating the binary mask image I_{B}, the inverse binary mask generating circuitry 450 can generate an inverse binary mask image I_{B-Inv}, defined as:

$${I}_{B - Inv} = \vert {I}_{B} - 1 \vert \qquad ( 2 )$$

The inverse binary mask image I_{B-Inv} has the same size as the input 2D projection image, but with ones at positions not covered by the collimator and zeros at positions where the collimator is applied.

Using the mask images I_{B} and I_{B-Inv}, the penumbra simulation circuitry 460 generate a penumbra-simulating image I_{s,e}, which includes simulated penumbra effects at the collimator edges. For example, the image I_{s,e} can be generated by applying a Gaussian blur to the input image as below:

$${I}_{s , e} = GaussianBlur ( I ⊙ {I}_{B - Inv} , σ ) ⊙ {I}_{B} \qquad ( 3 )$$

where ⊙ is element-wise multiplication, and σ is a collimator simulation parameter that defines the Gaussian kernel width. The collimator simulation parameter σ can be specified by the user, or randomly selected by the penumbra simulation circuitry 460 from a group of predetermined values.

The value of σ can be determined in various ways. For example, a physical line profile can be established based the edges of the simulated collimator, and σ can be experimentally tuned to match the established profile. Alternatively, the size of the penumbra can be calculated based on the collimator edges and the focal spot size through analytical methods.

Using the inverse binary mask image I_{B-Inv}, the training image combination circuitry 470 can generate a final training image I_{c} by integrating the input 2D projection image, the image I_{s, noise}, and the penumbra-simulating image I_{s,e} as follows:

$${I}_{c} = ( I ⊙ {I}_{B - Inv} ) + {I}_{s , noise} + {I}_{s , e} \qquad ( 4 )$$

In the example shown in FIG. 4, by using collimator simulation parameters, such as α and σ, experimentally tuned to match the imaging conditions of the input image I, the generated final image I_{c} can realistically simulate the collimator effects. Subsequently, the image I_{c} can be used as a training image for a neural network, to improve the robustness of the neural network to collimators.

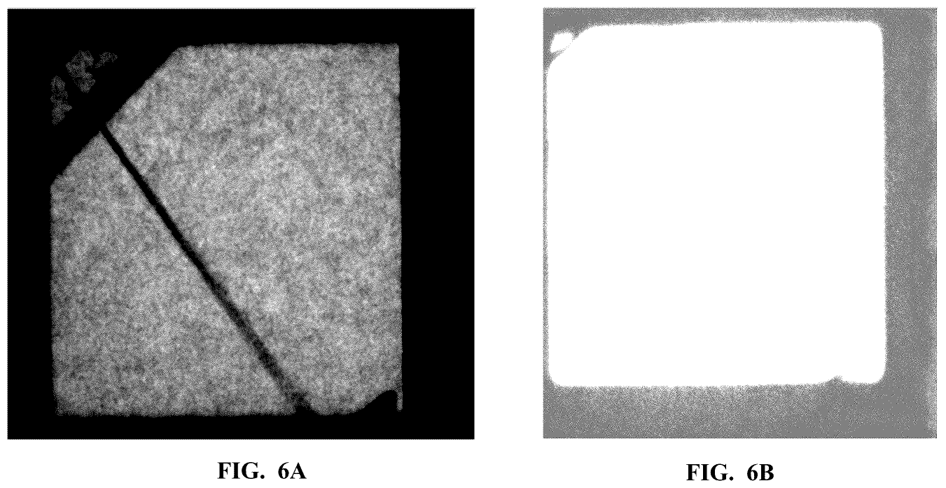

FIGS. 6A and 6B show images processed with a denoising neural network trained on images with collimator simulation, in accordance with embodiments of the disclosure. For comparison, the images are inferred from the same input image as FIGS. 1A and 1B, but denoised with a neural network trained on images with collimator simulation. The images in FIGS. 6A and 6B are displayed at different window levels. The comparison between FIGS. 1A-1B and 6A-6B demonstrates that artifacts due to inconsistencies in imaging conditions can be effectively suppressed by the collimator simulation method of the present disclosure.

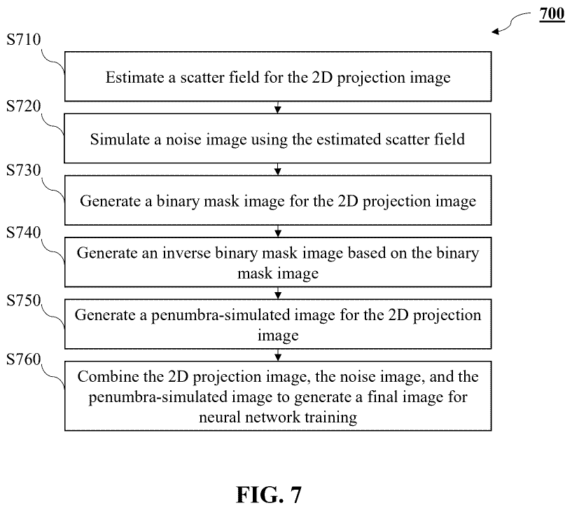

FIG. 7 shows a flow chart of an exemplary procedure 700 for generating a training image in accordance with embodiments of the disclosure. In step S710, a scatter field is estimated for a 2D projection image acquired without a collimator. In step S720, a noise image is generated based on the estimated scatter field to simulate the noise introduced by the scatter. In step S730, a binary mask image is generated for the 2D projection image. In step S730, an inverse binary mask image is generated based on the binary mask image. In step S750, a penumbra-simulating image is generated to simulate the increased signal along the collimator edges. In step S760, the 2D projection image, the noise image, and the penumbra-simulating image are combined to generate a final image for training the network.

The embodiments and examples describe above provide a method for generating a great number of collimated images from non-collimated images. This simple and efficient collimation simulation approach has been proven effective in eliminating image artifacts induced by collimators when AI-based image processing algorithms are pretrained on images without collimators. Moreover, this collimator simulation method can be sufficiently fast to be performed on-the-fly during the training of AI algorithms.

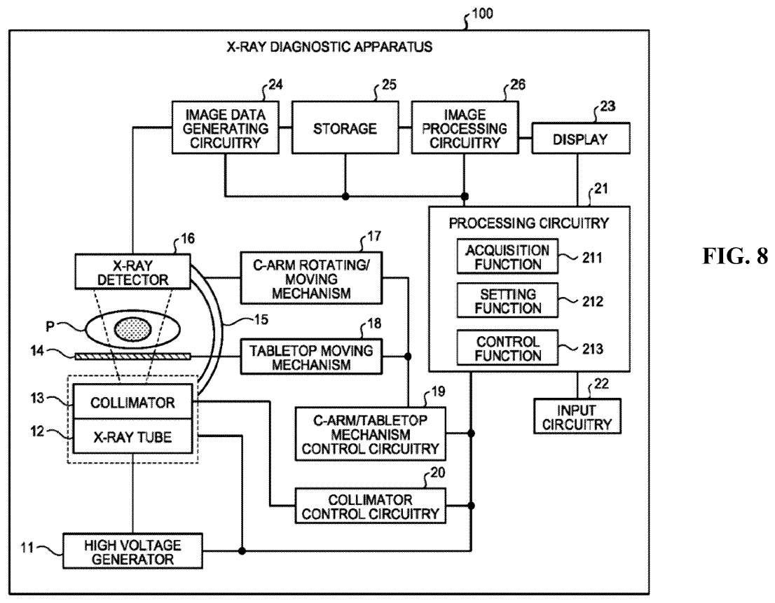

FIG. 8 is a diagram illustrating a configuration of an X-ray diagnostic apparatus 100 that can incorporate the techniques disclosed herein. As illustrated, the X-ray diagnostic apparatus 100 includes a high voltage generator 11, an X-ray tube 12, a collimator 13, a tabletop 14, a C-arm 15, an X-ray detector 16, a C-arm rotating/moving mechanism 17, a tabletop moving mechanism 18, C-arm/tabletop mechanism control circuitry 19, collimator control circuitry 20, processing circuitry 21, input circuitry 22, a display 23, image data generating circuitry 24, a storage 25, and image processing circuitry 26.

In the X-ray diagnostic apparatus 100, each processing function is stored in the storage 25 in the form of a computer program executable by a computer. The C-arm/tabletop mechanism control circuitry 19, the collimator control circuitry 20, the processing circuitry 21, the image data generating circuitry 24, and the image processing circuitry 26 are a processor that reads a computer program from the storage 25 and executes the computer program to implement the function corresponding to the computer program. In other words, each circuit in a state in which a computer program is read has the function corresponding to the read computer program.

The term “processor” used in the description above means, for example, a central processing unit (CPU), a graphics processing unit (GPU), or a circuit such as an application specific integrated circuit (ASIC) and a programmable logic device (for example, simple programmable logic device (SPLD), complex programmable logic device (CPLD), and a field programmable gate array (FPGA)). The processor reads and executes a computer program stored in the storage circuit to implement the function. The computer program may be directly built in a circuit in the processor, rather than being stored in a storage circuit. In this case, the processor implements the function by reading and executing the computer program built in the circuit. Each processor in the present embodiment may not be configured as a single circuit, but a plurality of independent circuits may be combined into a single processor, which implements the function.

The high voltage generator 11 generates high voltage and supplies the generated high voltage to the X-ray tube 12, under control by the processing circuitry 21. The X-ray tube 12 generates X-rays using the high voltage supplied from the high voltage generator 11.

The collimator 13 narrows X-rays produced by the X-ray tube 12 such that the X-rays are selectively applied to a region of interest of a subject P, under control by the collimator control circuitry 20. For example, the collimator 13 has four slidable collimator blades. The collimator 13 allows these collimator blades to slide under control by the collimator control circuitry 20 and thereby narrows the X-rays produced by the X-ray tube 12 to apply the narrowed X-rays to the subject P. The collimator 13 also includes an additional filter for adjusting the radiation quality. The additional filter is set, for example, depending on tests. The tabletop 14 is a bed on which the subject P lies and is disposed on a not-illustrated table (couch). The subject P is not included in the X-ray diagnostic apparatus 100.

The X-ray detector 16 detects X-rays transmitted through the subject P. For example, the X-ray detector 16 includes detecting elements arranged in a matrix. Each detecting element converts X-rays transmitted through the subject P into an electrical signal, accumulates the electrical signals, and transmits the accumulated electrical signals to the image data generating circuitry 24.

The C-arm 15 holds the X-ray tube 12, the collimator 13, and the X-ray detector 16. The C-arm 15 is rotated fast like a propeller around the subject P lying on the tabletop 14, by a motor provided at a support (not illustrated). Here, the C-arm 15 is rotatably supported with respect to three axes orthogonal to each other, namely, the XYZ axes, and is rotated individually in each axis by a not-illustrated driver. The X-ray tube 12 and the collimator 13 are disposed to be opposed to the X-ray detector 16 by means of the C-arm 15 with the subject P interposed. Although the X-ray diagnostic apparatus 100 is a single-plane system by way of example, embodiments are not limited thereto and may employ a biplane system.

The C-arm rotating/moving mechanism 17 is a mechanism for rotating and moving the C-arm 15. The C-arm rotating/moving mechanism 17 can also change a source image receptor distance (SID) which is the distance between the X-ray tube 12 and the X-ray detector 16. The C-arm rotating/moving mechanism 17 can also rotate the X-ray detector 16 held by the C-arm 15. The tabletop moving mechanism 18 is a mechanism for moving the tabletop 14.

The C-arm/tabletop mechanism control circuitry 19 controls the C-arm rotating/moving mechanism 17 and the tabletop moving mechanism 18 under control by the processing circuitry 21 to adjust the rotation and movement of the C-arm 15 and the movement of the tabletop 14. For example, the C-arm/tabletop mechanism control circuitry 19 controls rotation imaging to collect projection data at a predetermined frame rate while rotating the C-arm 15, under control by the processing circuitry 21. The collimator control circuitry 20 controls the radiation range of X-rays applied to the subject P by adjusting the aperture of the collimator blades of the collimator 13, under control by the processing circuitry 21.

The image data generating circuitry 24 generates projection data using the electrical signal obtained through conversion of X-rays by the X-ray detector 16 and stores the generated projection data into the storage 25. For example, the image data generating circuitry 24 performs current-voltage conversion, analog-digital (A/D) conversion, and parallel-serial conversion on the electrical signal received from the X-ray detector 16 to generate projection data. The image data generating circuitry 24 then stores the generated projection data into the storage 25.

The storage 25 accepts and stores the projection data generated by the image data generating circuitry 24. The storage 25 stores computer programs corresponding to various functions to be read and executed by the circuits illustrated. As an example, the storage 25 stores a computer program corresponding to an acquisition function 211, a computer program corresponding to a setting function 212, and a computer program corresponding to a control function 213 to be read and executed by the processing circuitry 21.

The image processing circuitry 26 performs various image processing on the projection data stored in the storage 25 to generate an X-ray image, under control by the processing circuitry 21 described later. Alternatively, the image processing circuitry 26 directly acquires projection data from the image data generating circuitry 24 and performs various image processing on the acquired projection data to generate an X-ray image, under control by the processing circuitry 21 described later. The image processing circuitry 26 may store the processed X-ray image into the storage 25. For example, the image processing circuitry 26 can execute various processing with image processing filters such as moving average (smoothing) filter, Gaussian filter, median filter, recursive filter, and bandpass filter.

The image processing circuitry 26 also forms reconstruction data (volume data) from projection data collected by rotation imaging. The image processing circuitry 26 then stores the reconstructed volume data into the storage 25. The image processing circuitry 26 generates a three-dimensional image from volume data. For example, the image processing circuitry 26 generates a volume rendering image or a multi planar reconstruction (MPR) image from volume data. The image processing circuitry 26 then stores the generated three-dimensional image into the storage 25. It is noted that the image processing circuitry 26 is an example of the reconstruction circuitry, which can be described or referenced in the claims.

The input circuitry 22 is implemented by, for example, a trackball, a switch button, a mouse, and a keyboard for setting a predetermined region (for example, a region of interest such as a section concerned), and a footswitch for emitting X-rays. The input circuitry 22 is connected to the processing circuitry 21 and converts an input operation accepted from the operator into an electrical signal for output to the processing circuitry 21. The display 23 displays a graphical user interface (GUI) for accepting the operator's instruction and a variety of images generated by the image processing circuitry 26.

The processing circuitry 21 controls the operation of the entire X-ray diagnostic apparatus 100. Specifically, the processing circuitry 21 executes various processing by reading a computer program corresponding to the control function 213 for controlling the entire apparatus from the storage 25 for execution. For example, the control function 213 controls an X-ray radiation dose to be applied to the subject P and ON/OFF by controlling the high voltage generator 11 in accordance with the operator's instruction forwarded from the input circuitry 22 and adjusting the voltage supplied to the X-ray tube 12. For example, the control function 213 controls the C-arm/tabletop mechanism control circuitry 19 in accordance with the operator's instruction and adjusts the rotation and movement of the C-arm 15 and the movement of the tabletop 14. For example, the control function 213 controls the radiation range of X-rays applied to the subject P by controlling the collimator control circuitry 20 in accordance with the operator's instruction and adjusting the aperture of the collimator blades of the collimator 13.

The control function 213 also controls, for example, the image data generation processing by the image data generating circuitry 24 and the image processing or the analysis processing by the image processing circuitry 26 in accordance with the operator's instruction. The control function 213 also performs control such that a GUI for accepting the operator's instruction or an image stored in the storage 25 appears on the display 23.

In an embodiment, the processing circuitry 21 executes the control function 213 described above as well as the acquisition function 211 and the setting function 212. It is noted that the processing circuitry 21 is an example of the processing circuitry in the claims.

Embodiments of collimator simulation approaches described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus, such as a networked device or server, user devices, and the like. The computer storage medium can be a machine-readable storage device, a machine-readable storage substrate, a random or serial access memory device, or a combination of one or more of them.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

According to an embodiment, the processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

In another embodiment, the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

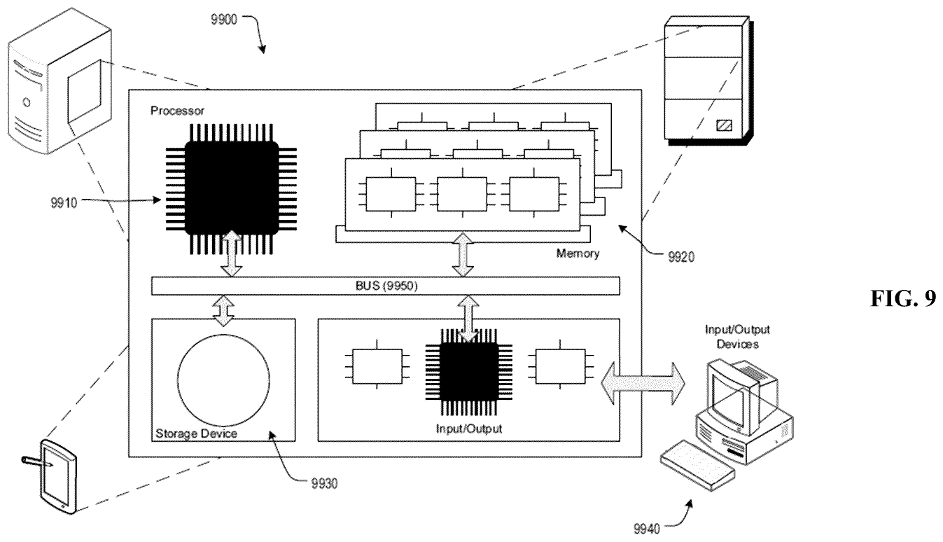

An example of a type of computer is shown in FIG. 9. The computer 9900 can be used for the operations described in association with any of the computer-implement methods described previously, according to one implementation. For example, the computer 9900 can be an example of devices 125, 135, 151, or a server (such as the server 190). The computer 9900 includes processing circuitry, as discussed above. The provider server 190 and the client server 191 can include other components not explicitly illustrated in FIG. 9 such as a CPU, GPU, frame buffer, etc. The processing circuitry includes one or more of the elements discussed next with reference to FIG. 9. In FIG. 9, the computer 9900 includes a processor 9910, a memory 9920, a storage device 9930, and an input/output device 9940. Each of the components 9910, 9920, 9930, and 9940 are interconnected using a system bus 9950. The processor 9910 is capable of processing instructions for execution within the system 9900. In one implementation, the processor 9910 is a single-threaded processor. In another implementation, the processor 9910 is a multi-threaded processor. The processor 9910 is capable of processing instructions stored in the memory 9920 or on the storage device 9930 to display graphical information for a user interface on the input/output device 9940.

The memory 9920 stores information within the computer 9900. In one implementation, the memory 9920 is a computer-readable medium. In one implementation, the memory 9920 is a volatile memory unit. In another implementation, the memory 9920 is a non-volatile memory unit.

The storage device 9930 is capable of providing mass storage for the computer 9900. In one implementation, the storage device 9930 is a computer-readable medium. In various different implementations, the storage device 9930 may be a floppy disk device, a hard disk device, an optical disk device, or a tape device.

The input/output device 9940 provides input/output operations for the computer 9900. In one implementation, the input/output device 9940 includes a keyboard and/or pointing device. In another implementation, the input/output device 9940 includes a display unit for displaying graphical user interfaces.

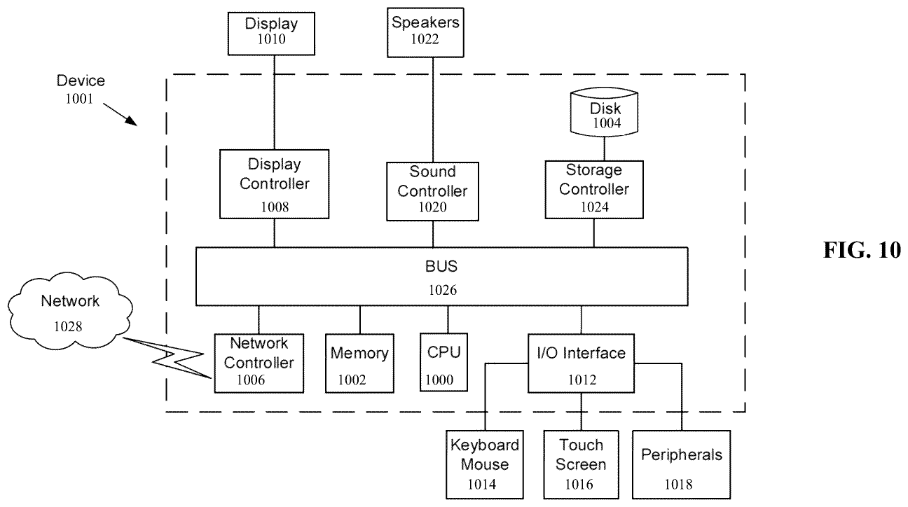

Next, a hardware description of a device according to the present embodiments is described with reference to FIG. 10. In FIG. 10, the device 1001, includes processing circuitry, as discussed above. The processing circuitry includes one or more of the elements discussed next with reference to FIG. 10. The device can include other components not explicitly illustrated in FIG. 10, such as a CPU, GPU, frame buffer, etc. In FIG. 10, the device includes a CPU 1000 which performs the processes described above/below. The process data and instructions may be stored in memory 1002. These processes and instructions may also be stored on a storage medium disk 1004 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 1001 communicates, such as a server or computer.

Further, the claimed advancements may be provided as a utility application, background daemon, or component of an operating system, or combination thereof, executing in conjunction with CPU 1000 and an operating system such as Microsoft Windows, UNIX, Solaris, LINUX, Apple MAC-OS and other systems known to those skilled in the art.

The hardware elements in order to achieve the device 1001 may be realized by various circuitry elements, known to those skilled in the art. For example, CPU 1000 may be a Xenon or Core processor from Intel of America or an Opteron processor from AMD of America, or may be other processor types that would be recognized by one of ordinary skill in the art. Alternatively, the CPU 1000 may be implemented on an FPGA, ASIC, PLD or using discrete logic circuits, as one of ordinary skill in the art would recognize. Further, CPU 1000 may be implemented as multiple processors cooperatively working in parallel to perform the instructions of the processes described above.

The device in FIG. 10 also includes a network controller 1006, such as an Intel Ethernet PRO network interface card from Intel Corporation of America, for interfacing with network 1028, and to communicate with the other devices. As can be appreciated, the network 1028 can be a public network, such as the Internet, or a private network such as an LAN or WAN network, or any combination thereof and can also include PSTN or ISDN sub-networks. The network 1028 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device further includes a display controller 1008, such as a NVIDIA Geforce GTX or Quadro graphics adaptor from NVIDIA Corporation of America for interfacing with display 1010, such as an LCD monitor. A general purpose I/O interface 1012 interfaces with a keyboard and/or mouse 1014 as well as a touch screen panel 1016 on or separate from display 1010. General purpose I/O interface also connects to a variety of peripherals 1018 including printers and scanners.

A sound controller 1020 is also provided in the device 1001 to interface with speakers/microphone 1022 thereby providing sounds and/or music.

The general purpose storage controller 1024 connects the storage medium disk 1004 with communication bus 1026, which may be an ISA, EISA, VESA, PCI, or similar, for interconnecting all of the components of the device. A description of the general features and functionality of the display 1010, keyboard and/or mouse 1014, as well as the display controller 1008, storage controller 1024, network controller 1006, sound controller 1020, and general purpose I/O interface 1012 is omitted herein for brevity as these features are known.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.
(1) A method for performing image processing in an X-ray imaging system, the X-ray imaging system having a collimator for collimating an incident direction of X-rays with respect to an X-ray detector, the method comprising: obtaining a two-dimensional (2D) projection image acquired by the X-ray imaging system; and inputting the obtained 2D projection image into a pretrained machine-learning model, to infer a 2D projection image from an output of the pretrained machine-learning model, the inferred 2D projection image having an image quality better than an image quality of the obtained 2D projection image; wherein the pretrained machine-learning model is trained on a plurality of training images, each training image of the plurality of training images being obtained by: receiving a first 2D projection image acquired by an X-ray imaging system under a condition that no collimator is applied for X-ray collimation, determining a collimator simulation parameter, estimating a scatter field for the first 2D projection image, generating a noise-simulating image based on the estimated scatter field, and generating a penumbra-simulating image to simulate a penumbra caused by a collimator, based on the determined collimator simulation parameter, and generating a second 2D projection image, as the training image, by combining the noise-simulating image, the penumbra-simulating image, and the first 2D projection image.
(2) The method of (1), wherein the determined collimator simulation parameter includes a scaling factor, and the step of estimating the scatter field further includes multiplying the scaling factor with a mean intensity of the first 2D projection image to obtain a constant scatter signal, as the estimated scatter field.
(3) The method of (1), wherein the step of estimating the scatter field further includes inputting the first 2D projection image into a second pretrained neural network and obtaining a scatter signal predicted by the second pretrained neural network, as the estimated scatter field.
(4) The method of (1), wherein the step of estimating the scatter field further includes, performing a Monte Carlo simulation based on the first 2D projection image and a 3D phantom to derive a scatter signal, as the estimated scatter field.
(5) The method of (1), wherein the step of estimating the scatter field further includes, solving a radiative transfer equation based on the first 2D projection image and a 3D phantom to derive a scatter signal, as the estimated scatter field.
(6) The method of (1), wherein the step of generating the noise-simulating image further includes retrieving, from a predetermined look-up table, an image corresponding to the estimated scatter field, as the generated noise-simulating image.
(7) The method of (6), wherein the retrieved image is predetermined to simulate noise caused by scatter corresponding to the estimated scatter field, as well as at least one of electronic noise, noise from a polychromatic beam, and noise caused by X-ray detector gain.
(8) The method of (1), wherein the step of generating the noise-simulating image further includes performing a Monte Carlo simulation using the estimated scatter field and the first 2D projection image to derive a noise image, as the generated noise-simulating image.
(9) The method of (8), wherein the derived noise image includes noise caused by the estimated scatter field, as well as at least one of electronic noise, noise from a polychromatic beam, and noise caused by X-ray detector gain.
(10) The method of (1), wherein the determined collimator simulation parameter includes a collimator geometry parameter indicating a percentage of the first 2D projection image to be collimated.
(11) The method of (10), wherein the determined collimator simulation parameter further includes another collimator geometry parameter indicating a collimation aspect ratio between a row direction and a column direction of the X-ray detector.
(12) The method of (10), wherein the determined collimator simulation parameter further includes a Gaussian kernel width, and the step of generating the penumbra-simulating image further includes: generating a first binary mask image, based on the collimator geometry parameter, generating a second binary mask image, the second binary mask image being an inverse of the first binary mask image, and applying a Gaussian blur to the first 2D projection image, based on the Gaussian kernel width, the first binary mask image, and the second binary mask image.
(13) The method of (1), wherein the step of determining the collimator simulation parameter includes receiving a collimator simulation parameter specified by a user, or selecting a collimator simulation parameter from a set of predetermined collimator simulation parameters, as the determined collimator simulation parameter.
(14) The method of (1), wherein both the noise-simulating image and the penumbra-simulating image have a same size as the first 2D projection image.
(15) The method of (1), wherein the X-ray imaging system is a 2D projection X-ray imaging system, a Computed Tomography (CT) imaging system, or a C-arm interventional imaging system.
(16) An apparatus for performing image data processing in an X-ray imaging system, the X-ray imaging system having a collimator for collimating an incident direction of X-rays with respect to an X-ray detector, the apparatus comprising processing circuitry configured to: obtain a two-dimensional (2D) projection image acquired by the X-ray imaging system; and input the obtained 2D projection image into a pretrained machine-learning model, to infer a 2D projection image from an output of the pretrained machine-learning model, the inferred 2D projection image having an image quality better than an image quality of the obtained 2D projection image, wherein the pretrained machine-learning model is trained on a plurality of training images, each training image of the plurality of training images being obtained by: receiving a first 2D projection image acquired by an X-ray imaging system under a condition that no collimator is applied for X-ray collimation, determining a collimator simulation parameter, estimating a scatter field for the first 2D projection image, generating a noise-simulating image based on the estimated scatter field, and generating a penumbra-simulating image to simulate a penumbra caused by a collimator, based on the determined collimator simulation parameter, and generating a second 2D projection image, as the training image, by combining the noise-simulating image, the penumbra-simulating image, and the first 2D projection image.
(17) The apparatus of (16), wherein the determined collimator simulation parameter includes a scaling factor, and the step of estimating the scatter field further includes multiplying the scaling factor with a mean intensity of the first 2D projection image to obtain a constant scatter signal, as the estimated scatter field.
(18) The apparatus of (16), wherein the step of generating the noise-simulating image further includes retrieving, from a predetermined look-up table, an image corresponding to the estimated scatter field, as the generated noise-simulating image.
(19) The apparatus of (16), wherein the determined collimator simulation parameter includes a collimator geometry parameter indicating a percentage of the first 2D projection image to be collimated.
(20) The apparatus of (19), wherein the determined collimator simulation parameter further includes a Gaussian kernel width, and the step of generating the penumbra-simulating image further includes: generating a first binary mask image, based on the collimator geometry parameter, generating a second binary mask image, the second binary mask image being an inverse of the first binary mask image, and applying a Gaussian blur to the first 2D projection image, based on the Gaussian kernel width, the first binary mask image, and the second binary mask image.

Numerous modifications and variations of the embodiments presented herein are possible in light of the above teachings. It is therefore to be understood that within the scope of the claims, the disclosure may be practiced otherwise than as specifically described herein.

What is claimed is:

1. A method for performing image processing in an X-ray imaging system, the X-ray imaging system having a collimator for collimating an incident direction of X-rays with respect to an X-ray detector, the method comprising:
obtaining a two-dimensional (2D) projection image acquired by the X-ray imaging system; and
inputting the obtained 2D projection image into a pretrained machine-learning model, to infer a 2D projection image from an output of the pretrained machine-learning model, the inferred 2D projection image having an image quality better than an image quality of the obtained 2D projection image; wherein
the pretrained machine-learning model is trained on a plurality of training images, each training image of the plurality of training images being obtained by:
receiving a first 2D projection image acquired by an X-ray imaging system under a condition that no collimator is applied for X-ray collimation,
determining a collimator simulation parameter,
estimating a scatter field for the first 2D projection image, generating a noise-simulating image based on the estimated scatter field, and generating a penumbra-simulating image to simulate a penumbra caused by a collimator, based on the determined collimator simulation parameter, and
generating a second 2D projection image, as the training image, by combining the noise-simulating image, the penumbra-simulating image, and the first 2D projection image.

2. The method of claim 1, wherein the determined collimator simulation parameter includes a scaling factor, and the step of estimating the scatter field further includes multiplying the scaling factor with a mean intensity of the first 2D projection image to obtain a constant scatter signal, as the estimated scatter field.

3. The method of claim 1, wherein the step of estimating the scatter field further includes inputting the first 2D projection image into a second pretrained neural network and obtaining a scatter signal predicted by the second pretrained neural network, as the estimated scatter field.

4. The method of claim 1, wherein the step of estimating the scatter field further includes, performing a Monte Carlo simulation based on the first 2D projection image and a 3D phantom to derive a scatter signal, as the estimated scatter field.

5. The method of claim 1, wherein the step of estimating the scatter field further includes, solving a radiative transfer equation based on the first 2D projection image and a 3D phantom to derive a scatter signal, as the estimated scatter field.

6. The method of claim 1, wherein the step of generating the noise-simulating image further includes retrieving, from a predetermined look-up table, an image corresponding to the estimated scatter field, as the generated noise-simulating image.

7. The method of claim 6, wherein the retrieved image is predetermined to simulate noise caused by scatter corresponding to the estimated scatter field, as well as at least one of electronic noise, noise from a polychromatic beam, and noise caused by X-ray detector gain.

8. The method of claim 1, wherein the step of generating the noise-simulating image further includes performing a Monte Carlo simulation using the estimated scatter field and the first 2D projection image to derive a noise image, as the generated noise-simulating image.

9. The method of claim 8, wherein the derived noise image includes noise caused by the estimated scatter field, as well as at least one of electronic noise, noise from a polychromatic beam, and noise caused by X-ray detector gain.

10. The method of claim 1, wherein the determined collimator simulation parameter includes a collimator geometry parameter indicating a percentage of the first 2D projection image to be collimated.

11. The method of claim 10, wherein the determined collimator simulation parameter further includes another collimator geometry parameter indicating a collimation aspect ratio between a row direction and a column direction of the X-ray detector.

12. The method of claim 10, wherein the determined collimator simulation parameter further includes a Gaussian kernel width, and the step of generating the penumbra-simulating image further includes:
generating a first binary mask image, based on the collimator geometry parameter,
generating a second binary mask image, the second binary mask image being an inverse of the first binary mask image, and
applying a Gaussian blur to the first 2D projection image, based on the Gaussian kernel width, the first binary mask image, and the second binary mask image.

13. The method of claim 1, wherein the step of determining the collimator simulation parameter includes receiving a collimator simulation parameter specified by a user, or selecting a collimator simulation parameter from a set of predetermined collimator simulation parameters, as the determined collimator simulation parameter.

14. The method of claim 1, wherein both the noise-simulating image and the penumbra-simulating image have a same size as the first 2D projection image.

15. The method of claim 1, wherein the X-ray imaging system is a 2D projection X-ray imaging system, a Computed Tomography (CT) imaging system, or a C-arm interventional imaging system.

16. An apparatus for performing image data processing in an X-ray imaging system, the X-ray imaging system having a collimator for collimating an incident direction of X-rays with respect to an X-ray detector, the apparatus comprising:
processing circuitry configured to
obtain a two-dimensional (2D) projection image acquired by the X-ray imaging system; and
input the obtained 2D projection image into a pretrained machine-learning model, to infer a 2D projection image from an output of the pretrained machine-learning model, the inferred 2D projection image having an image quality better than an image quality of the obtained 2D projection image, wherein
the pretrained machine-learning model is trained on a plurality of training images, each training image of the plurality of training images being obtained by:
receiving a first 2D projection image acquired by an X-ray imaging system under a condition that no collimator is applied for X-ray collimation,
determining a collimator simulation parameter,
estimating a scatter field for the first 2D projection image, generating a noise-simulating image based on the estimated scatter field, and generating a penumbra-simulating image to simulate a penumbra caused by a collimator, based on the determined collimator simulation parameter, and
generating a second 2D projection image, as the training image, by combining the noise-simulating image, the penumbra-simulating image, and the first 2D projection image.

17. The apparatus of claim 16, wherein the determined collimator simulation parameter includes a scaling factor, and the step of estimating the scatter field further includes multiplying the scaling factor with a mean intensity of the first 2D projection image to obtain a constant scatter signal, as the estimated scatter field.

18. The apparatus of claim 16, wherein the step of generating the noise-simulating image further includes retrieving, from a predetermined look-up table, an image corresponding to the estimated scatter field, as the generated noise-simulating image.

19. The apparatus of claim 16, wherein the determined collimator simulation parameter includes a collimator geometry parameter indicating a percentage of the first 2D projection image to be collimated.

20. The apparatus of claim 19, wherein the determined collimator simulation parameter further includes a Gaussian kernel width, and the step of generating the penumbra-simulating image further includes:
generating a first binary mask image, based on the collimator geometry parameter,
generating a second binary mask image, the second binary mask image being an inverse of the first binary mask image, and
applying a Gaussian blur to the first 2D projection image, based on the Gaussian kernel width, the first binary mask image, and the second binary mask image.
